(19) United States
(12) Patent Application Publication
(10) Pub. No.: US 2013/0090671 A1
(43) Pub. Date: Apr. 11, 2013

(54) ENDOSCOPE SUTURING DEVICE

(71) Applicants: Sang Su Yuk, Daegu (KR); Keun Yong Yuk, Daegu (KR); Kyo Sik Hwang, Daegu (KR)

(72) Inventors: Sang Su Yuk, Daegu (KR); Keun Yong Yuk, Daegu (KR); Kyo Sik Hwang, Daegu (KR)

(73) Assignees: Keun Yong YUK, Daegu (KR); Sang Su YUK, Daegu (KR)

(21) Appl. No.: 13/703,623

(22) PCT Filed: May 12, 2011
(86) PCT No.: PCT/KR2011/003495

§ 371 Date: Dec. 11, 2012

(30) Foreign Application Priority Data
Jun. 18, 2010 (KR) 10-2010-0058108

(51) Int. Cl. A61B 17/04

(52) U.S. Cl. CPC A61B 17/0469
USPC 606/145

(57) ABSTRACT

An endoscopic suturing device for suturing a surgical site in the body using an endoscope. Each of first, second and third modules (100, 200, 300) has a needle which sutures the surgical site at the distal end thereof, a clamp which holds a suturing thread of the needle, and a clamp and cutter which cuts the suturing thread of the needle, and in which each of the first, second and third modules is rotated upward and downward and tilted forward and backward and to the left and right by a location control means (500) in order to adjust the location thereof. It is possible to simply move the modules to a suturing site and to simplify the operation of suturing the surgical site using the needle or the operation of binding and fixing the surgical thread and cutting the surgical thread, thereby making surgical operability excellent.

### TECHNICAL FIELD

The present invention relates to an endoscopic suturing device for suturing a surgical site in the body using an endoscope. More particularly, the present invention relates to an endoscopic suturing device, in which each of first, second and third modules has a needle which sutures the surgical site at the distal end thereof, a clamp which holds a suturing thread of the needle, and a clamp and cutter which cuts the suturing thread of the needle, and in which each of the first, second and third modules is rotated upward and downward and tilted forward and backward and to the left and right by a location control means in order to adjust the location thereof. It is possible to simply move the modules to a suturing site and to simplify the operation of suturing the surgical site using the needle or the operation of binding and fixing the surgical thread and cutting the surgical thread, thereby making surgical operability excellent.

### BACKGROUND ART

These days, for an incision operation on a human body, precise surgical methods using an endoscope are generally used, which minimizes the size of incision required.

In particular, such an endoscope surgery has a variety of advantages. For example, it reduces the pain of patients and enables patients to recover more rapidly.

The endoscope surgery is carried out by inserting a mechanical device into a targeted region inside the body in order to incise and suture a targeted region while observing the targeted region using the endoscope, which has a lens or a camera.

For example, Korean Laid-On Patent Application Nos. 10-2010-0000291 and 10-2006-0092831 and Korean Patent No. 10-0531496 disclose a variety of surgical suturing devices. These devices of the related art have problems in that the control over the movement of such a device to a suturing region is very difficult, and the operation of suturing the suturing region of the body by stitching it using a suturing thread as well as the operation of binding and fixing the suturing wire or cutting the suturing wire is very difficult. These problems consequently lower the operability of the surgery.

Furthermore, the lowered operability of the surgery results in several problems. For example, the surgery requires skillfulness, and the surgery time is increased, which increases the pain of the patient.

### DISCLOSURE

### Technical Problem

Accordingly, the present invention has been made keeping in mind the above problems occurring in the related art, and is intended to provide an endoscopic suturing device, in which a needle which sutures the surgical site at the distal end thereof, a clamp which holds a suturing thread of the needle, and a clamp and cutter which cuts the suturing thread of the needle are rotated upward and downward and tilted forward and backward and to the left and right using a location control means is order to adjust the location thereof. It is possible to simply move the modules to a suturing site and to simplify the operation of suturing the surgical site using the needle or the operation of binding and fixing the surgical thread and cutting the surgical thread, thereby making surgical operability excellent.

The present invention is also intended to simplify the surgical operation and reduce the operation time due to excellent operability, thereby minimizing the pain of the patient and improving the reliability of the operation.

The present invention is also intended to improve patient satisfaction with regard to the operation outcome by effectively stitching and suturing the suturing site using the suturing thread.

### Technical Solution

According to an aspect of the present invention, provided is an endoscopic suturing device for suturing a surgical site in a human body using an endoscope. The endoscopic suturing device includes a first module disposed inside a tubular body which is insertable into the human body, the first module being rotatable upward and backward and tilting-adjustable forward and backward and to left and right, and having a needle which stitches a suturing site; a second module being rotatable upward and backward and tilting-adjustable forward and backward and to left and right, and having a clamp which holds and binds a stitching fiber; and a third module being rotatable upward and backward and tilting-adjustable forward and backward and to left and right, and having a cutter and clamp which holds and cuts the suturing thread.

Each of the first, second and third modules may include a position control means which controls each of the first, second and third modules to rotate upward and downward and tilt forward and backward and to left and right toward the suturing site. The position control means includes a first lever, a second lever and a third lever which are connected together. The first lever in axially coupled to a motor shaft of a first motor which is horizontally disposed on a central cylinder so as to rotate upward and downward. The second and third levers are axially coupled to motor shafts of second and third motors which are vertically disposed and being positioned so as to perpendicularly cross each other. The second and third levers tilt forward and backward and to left and right.

The first, second and third levers may be connected by the cylinder and ball joints, respectively, noes to enable a combined movement of rotation and forward, backward, leftward and rightward tilting.

Each of the first, second and third motors may be configured as a direct current (DC) reduction motor including a reducer, a motor and an encoder which are coupled together.

The first module may be configured such that the rear end of the needle and one section of a lever are connected in the lower section of the cylinder so as to be rotatable about a hinge, the needle having a pinhole in a leading end through which the suturing thread is inserted, and that the other section of the lever and an actuator in an upper portion of the cylinder are connected to each other using a wire, so that the needle sutures the suturing site while being rotated about the hinge by the actuator.

The second module may include a fixing member and a pressing member which are disposed with an “X” shape in the lower portion of the cylinder. The rear end of the pressing member is connected to the actuator using a wire, and middle portions of the fixing member and the pressing member are hinge-coupled to each other. The second module may hold the suturing thread in response to the actuator.

The third module may include a fixing member and a pressing member which are disposed with an “X” shape, in the lower portion of the cylinder. The fixing member integrally have a fixing blade in the front portion and a fixing portion in the rear portion. The rear end of the pressing member is connected to the actuator using a wire. The pressing member integrally has a cutting blade in the front portion and a pressing portion in the rear portion. The third module holds or cuts the suturing thread from front and back in response to the actuator.

A pressing piece which elastically holds the suturing thread may be provided in the pressing member of the second module and in the pressing portion of the pressing member of the third module. The pressing piece is elastically forced by a spring so as to protrude from a recess.

### Advantageous Effects

As described above, each of the first, second and third modules has the needle which sutures the surgical site at the distal end thereof, the clamp which holds the suturing thread of the needle, and the clamp and cutter which cuts the suturing thread of the needle. Each module is rotated upward and downward and tilted forward and backward and to the left and right using the location control means in order to adjust the location thereof. It is possible to simply move the modules to a suturing site and to simplify the operation of suturing the surgical site using the needle or the operation of binding and fixing the surgical thread and cutting the surgical thread, thereby making surgical operability excellent. It is also possible to simplify the surgical operation and reduce the operation time due to excellent operability, thereby minimizing the pain of the patient and improving the reliability of the operation.

### DESCRIPTION OF DRAWINGS

FIG. 1 is a conceptual view Showing the cross-sectional arrangement structure of a device according to the present invention;

FIG. 2 is a front confighratibn view showing the first module of the device according to the present invention;

FIG. 3 is a view snowing key parts of FIG. 2;

FIG. 4 is a front configuration view showing the second module of the device according to the present invention;

FIG. 5 is view showing key parts of FIG. 4;

FIG. 6 is a front configuration view showing the third module of the device according to the present invention;

FIG. 7 is a view showing key parts of FIG. 6;

FIG. 8 is a perspective view showing a position control means of the device according to the present invention;

FIG. 9 is a plan view showing key parts of FIG. 8;

FIG. 10 to FIG. 15 are sequential views showing the suturing process using the device according to the present invention.

| | |
|---|---|
| 2: tubular body | 10: needle |
| 100: first module | 200: second module |
| 300: third module | 20: clamp |
| 30: cutter and clamp | 500: position control means |
| 3: cylinder | 512: first lever |
| 522: second lever | 532: third lever |
| 11: hinge | 12: pinhole |
| 50: actuator | 51: wire |
| 210: fixing member | 220: pressing member |
| 310: fixing member | 311: fixing blade |
| 312: fixing portion | 320: pressing member |
| 321: cutting blade | 322: pressing portion |
| 60: pressing piece | 65: spring |

### BEST MODE

Hereinafter, exemplary embodiments of the present invention will be described in detail with reference to the accompanying drawings.

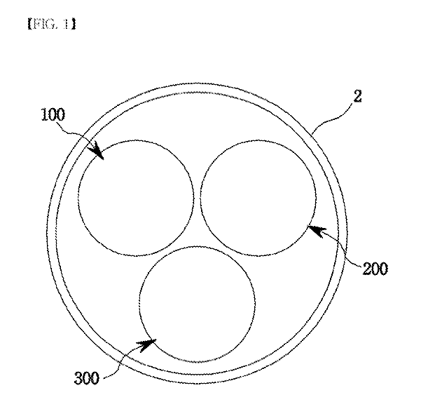

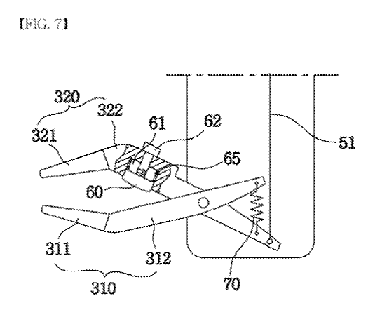

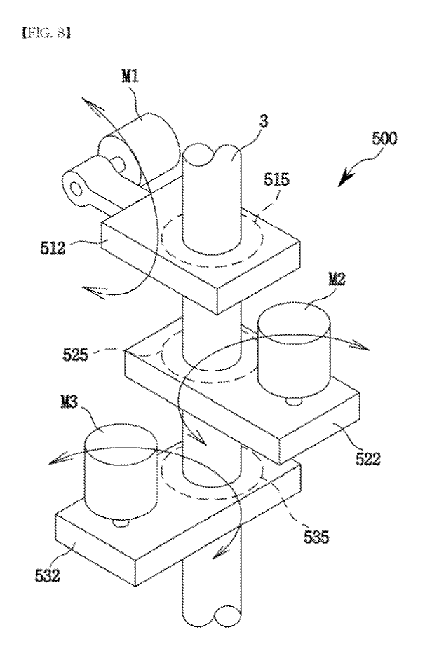

As shown in FIG. 1 to FIG. 9, an endoscope suturing device according to the present invention includes a tubular body 2 which can be inserted into the human body.

A first module 100 is disposed inside the tubular body. The first module 100 is rotatable upward and backward, is tilting-adjustable forward and backward and to the left and right, and has a needle 20 which stitches a suturing site.

A second module 200 is rotatable upward and backward, is tilting-adjustable forward and backward and to the left and right, and has a clamp 20 which holds and binds a stitching fiber.

A third module 300 is rotatable upward and backward, is tilting-adjustable forward and backward and to the left and right, and has a cutter and clamp 30 which holds and cuts the suturing thread.

Here, it is of course possible to use the first, second and third modules 100, 200 and 300 by independently inserting each of the modules 100, 200 and 300 into a separate tubular body 2.

That is, the first module 100, the second module 200 and the third module 300 can be individually provided and be independently inserted into the human body in order to carry out a suturing operation.

In addition, each of the first, second and third modules 100, 200 and 300 includes a position control means 500 which controls upward and downward rotation as well as tilting forward and backward and to the left and right toward the suturing site.

The position control means 500 is configured by connecting a first lever 512, a second lever 522 and a third lever 532 together. The first lever 512 is axially coupled to a motor shaft of a first motor M1 which is horizontally disposed on a central cylinder 3, such that it rotates upward and downward. The second and third levers 522 and 532 are axially coupled to motor shafts of second and third motors M2 and M3 which are vertically disposed, are positioned so as to perpendicularly cross each other, and tilt forward and backward and to the left and right.

Here, the first, second and third levers 512, 522 and 532 are connected by the cylinder 3 and ball tints 515, 525 and 535, respectively, such that they enable a movement in which rotation and forward, backward, leftward and rightward tilting are combined.

In addition, it is preferred that the first, second and third motors M1, M2 and M3 be applied as a direct current (DC) reduction motor in which a reducer, a motor and en encoder are coupled.

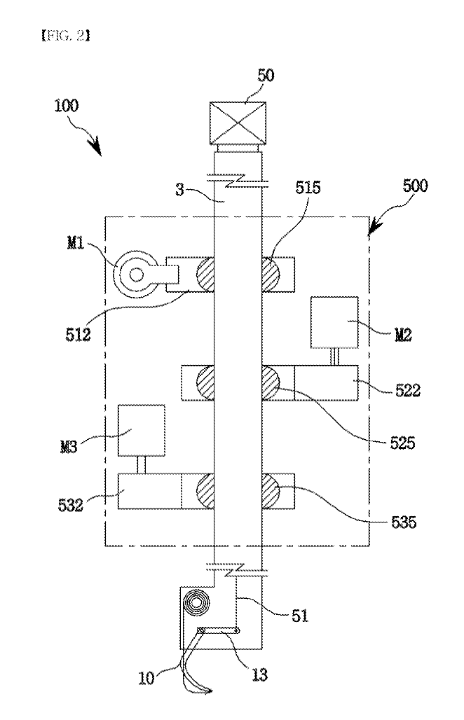

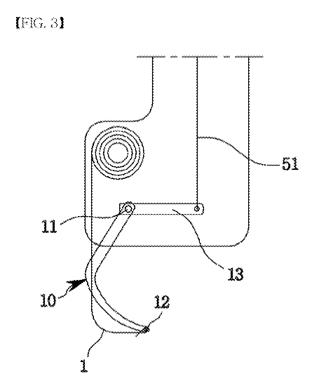

In addition, as shown in FIG. 2 and FIG. 3, the first module 100 is configured such that the rear end of the needle 10 and one section of a lever 13 are connected in the lower section of the cylinder such that they can rotate about a hinge 11. The needle 10 has a pinhole 12 in the leading end through which the suturing thread is inserted. The other section of the lever and an actuator 50 in the upper portion of the cylinder 3 are connected to each other using a wire 51. With this configuration, the needle sutures the suturing site while being rotated about the hinge by the actuator.

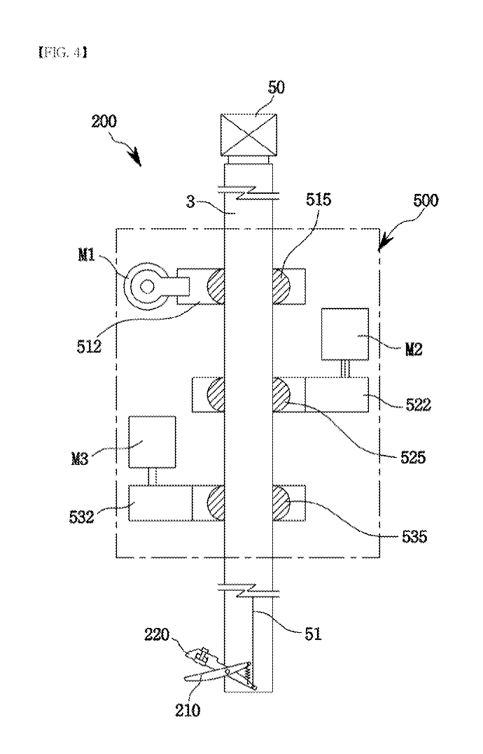

As shown in FIG. 4 and FIG. 5, the second module 200 includes a fixing member 210 and a pressing member 220 which are disposed with the “X” shape in the lower portion of the cylinder. The rear end of the pressing member 220 is connected to the actuator 50 using a wire 51. The middle portions of the fixing member 210 and the pressing member 220 are hinge-coupled to each other. With this configuration, the second module 200 holds the suturing thread in response to the actuator.

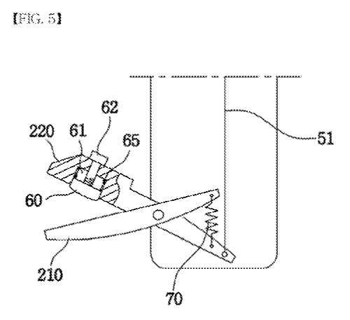

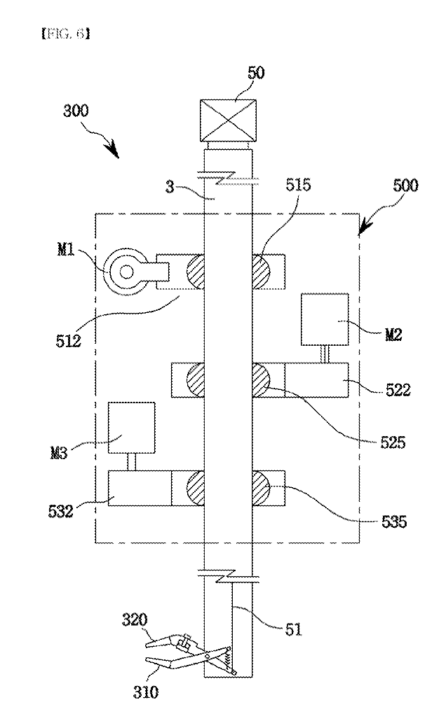

As shown in FIG. 6 and FIG. 7 the third module 300 includes a fixing member 310 and a pressing member 320 which are disposed with the “X” shape in the lower portion of the cylinder. The fixing member 310 integrally has a fixing blade 311 in the front portion and a fixing portion 312 in the rear portion. The rear end of the pressing member 320 is connected to the actuator 50 using a wire 51. The pressing member 320 integrally has a cutting blade 321 is the front portion and a pressing portion 322 in the rear portion. With this configuration, the third module 300 holds or cuts the suturing thread from the front and back in response to the actuator.

In addition, a pressing piece 60 which elastically holds the suturing thread is provided in the pressing member 220 of the second module 200 and in the pressing portion 322 of the pressing member 320 of the third module 300. The pressing piece 60 is elastically forced by a spring 65 so as to protrude from a recess 61.

As for reference numerals which have not been described, 62 indicates a support bolt which supports the pressing piece 60, and 70 indicates a return spring.

A description will be given below of the operation and function of the present invention which has the above-described configuration.

First, the device of the present invention is set in position by inserting it into a suturing site G in the human body.

In this state, each of the first, second and third modules 100, 200 and 300 inside the tubular body 2 is observed using an endoscope, and is moved to the suturing site G by rotating it upward and backward and tilting it forward and backward and to the left and right using the position control means 500.

This is possible since the respective central cylinder 3 of the first, second and third modules is controlled using the first lever 512 coupled to the motor shaft of the first motor M1 so that the cylinder rotates upward and downward, and since the respective central cylinder 3 of the first, second and third modules is controlled using the second and third levers 522 and 532 which are respectively coupled to the motor shafts of the second and third motors M2 and M3 and are oriented so as to perpendicularly cross each other so that the cylinder tilts forward and backward and to the left and right.

That is, the central cylinders 3 of the first, second and third modules are respectively moved to the suturing site G in a simple fashion by rotating them upward and downward and tilting them forward and backward and to the left and right using the first, second and third levers 512, 522 and 532 in response to actuation control over the first, second and third motors M1, M2 and M3.

In this state in which the first, second and third modules are positioned at the suturing site, the operation of suturing the suturing site G using the suturing thread 1 is performed by individually manipulating the first, second and third modules 100, 200 and 300.

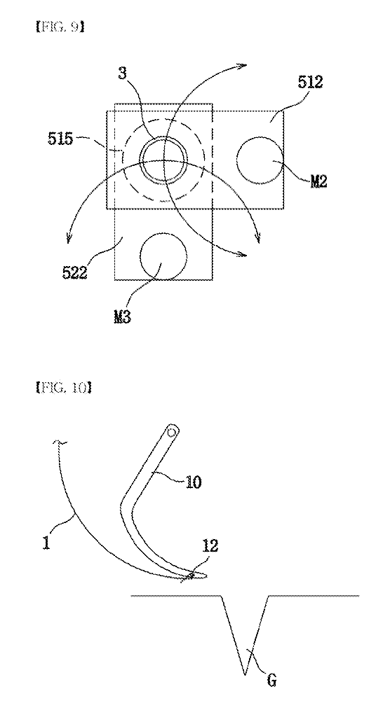

In more detail, in the state in which the needle 10 of the first module is controlled so as to be positioned at one portion of the suturing site, the wire 51 is pulled by the actuator 50, so that the end portion of the curved needle 10 crosses the suturing site G while rotating about the hinge, as shown in FIG. 11.

In this state, the suturing thread 1 is threaded through the pinhole 12 at the distal end of the needle 10.

After the and portion of the needle 10 has crossed the suturing site, position control is performed so that the suturing thread 1 at the end portion of the needle is positioned between the fixing member 210 and pressing member 220 of the second module 200.

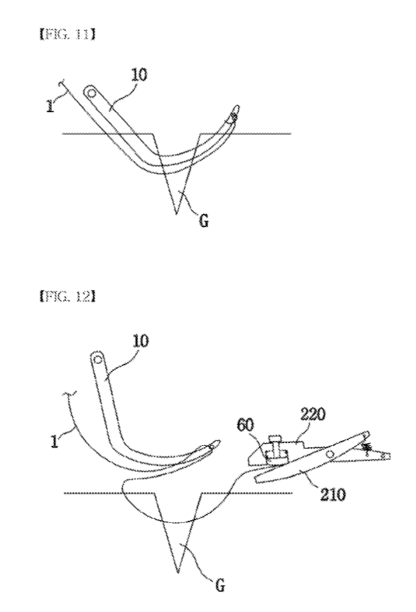

In addition, since the wire 51 is pulled by the actuator 50 of the second module, the pressing member 220 holds the end portion of the suturing thread 1 while rotating about the hinge, as shown in FIG. 12.

Here, the pressing member 220 firmly holds the suturing thread 1 as the pressing piece 60 is elastically forced thereto by the spring 65.

When the second module holds the suturing thread 1, the actuator 50 of the first module returns to the original position, so that the curved needle 10 returns to the original position.

Here, the suturing thread 1 is in the state of being inserted into the suturing site across it.

In this state, the first module is moved upward and forward by the position control means 500 and is located at the upper position in which the suturing thread 1 is inserted into the suturing site across it.

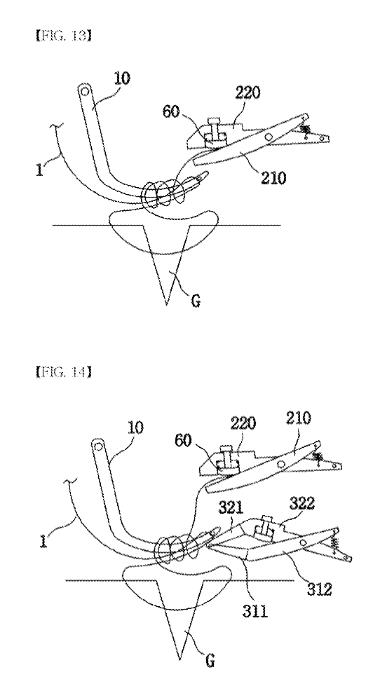

In sequence, as shown in FIG. 13, the second module 200 is rotated by the position control means 500 so as to wind the suturing thread 1 around the outer circumference of the curved needle 10 while holding the distal end of the suturing thread.

This is possible since the position control means causes the second module 200 to perform a combined movement of rotating upward and downward and tilting forward and backward and to the left and right.

In this state, position control is performed so that the suturing thread 1 at the end portion of the needle is positioned between the fixing member 310 and pressing member 320 of the second module 200 of the third module 300.

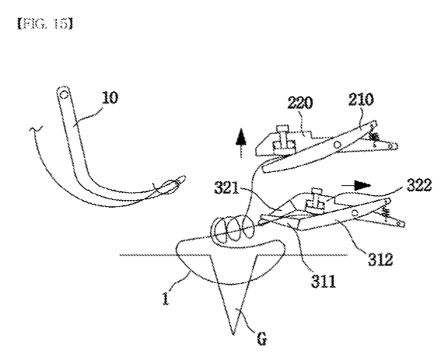

Since the wire 51 is pulled by the actuator 50 of the third module, the cutting blade 321 at the leading end of the pressing member and the fixing blade 311 at the leading end of the fixing member act as scissors having the “X” shape, thereby cutting a portion of the suturing thread 1 that has passed through the pinhole 12, while the pressing member 320 is rotating about the hinge, as shown in FIG. 14. Then, the cut suturing thread 1 is pressed and fixed by the pressing portion 322 of the pressing member and the fixing portion 312 of the fixing member.

Here, both ends of the cut suturing thread 1 are in the state of being held by the second and third modules 200 and 300.

In this state, as shown in FIG. 15, the cut suturing thread 1 is bound and fixed in the threaded state by pulling both ends of the cut suturing thread by moving the second and third modules 200 and 300 in the opposite directions.

In this way, the suturing site G is sutured by weaving one suturing thread 1 thereto.

In addition, the suturing site is sutured by repeating the process of suturing the suturing site by moving the first, second and third modules 100, 200 and 300 to the next region of the suturing site to which the suturing thread is weaved using the position control means 500.

### MODE FOR INVENTION

According to the present invention, each of the first, second and third modules has the needle which sutures the surgical site at the distal end thereof, the clamp which holds the suturing thread of the needle, and the clamp and cutter which cuts the suturing thread of the needle. Each module is rotated upward and downward and tilted forward and backward and to the left and right using the location control means in order to adjust the location thereof. It is possible to simply move the modules to a suturing site and to simplify the operation of suturing the surgical site using the needle or the operation of binding and fix in the surgical thread and cutting the surgical thread, thereby making surgical operability excellent.

1. An endoscopic suturing device for suturing a surgical site in a human body using an endoscope, the device comprising:
a first module disposed inside a tubular body which is insertable into the human body, the first module being rotatable upward and backward and tilting-adjustable forward and backward and to left and right, and having a needle which stitches a suturing site;
a second module being rotatable upward and backward and tilting-adjustable forward and backward and to left and right, and having a clamp which holds and binds a stitching fiber; and
a third module being rotatable upward and backward and tilting-adjustable forward and backward and to left and right, and having a cutter and clamp which holds and cuts the suturing thread.

2. The endoscopic suturing device of claim 1, wherein each of the first, second and third modules comprises a position control means which controls each of the first, second and third modules to rotate upward and downward and tilt forward and backward and to left and right toward the suturing site, wherein the position control means includes a first lever, a second lever and a third lever which are connected together, the first lever being axially coupled to a motor shaft of a first motor which is horizontally disposed on a central cylinder so as to rotate upward and downward, the second and third levers being axially coupled to motor shafts of second and third motors which are vertically disposed and being positioned so as to perpendicularly cross each other, the second and third levers tilting forward and backward and to left and right.

3. The endoscopic suturing device of claim 2, wherein the first, second and third levers are connected by the cylinder and ball joints, respectively, so as to enable a combined movement of rotation and forward, backward, leftward and rightward tilting.

4. The endoscopic suturing device of claim 2, wherein each of the first, second and third motors comprises a direct current reduction motor including a reducer, a motor and an encoder which are coupled together.

5. The endoscopic suturing device of claim 1, wherein the first module configured such that a rear end of the needle and one section of a lever are connected in a lower section of the cylinder so as to be rotatable about a hinge, the needle having a pinhole in a leading end through which the suturing thread is inserted, and that the other section of the lever and an actuator in an upper portion of the cylinder are connected to each other using a wire, so that the needle sutures the suturing site while being rotated about the hinge by the actuator.

6. The endoscopic suturing device of claim 1, wherein the second module comprises a fixing member and a pressing member which are disposed with an “X” shape in a lower portion of the cylinder, a rear end of the pressing member being connected to the actuator using a wire, and middle portions of the fixing member and the pressing member being hinge-coupled to each other, wherein the second module holds the suturing thread in response to the actuator.

7. The endoscopic suturing device of claim 1, wherein the third module comprises a fixing member and a pressing member which are disposed with an “X” shape in a lower portion of the cylinder, the fixing member integrally having a fixing blade in a front portion and a fixing portion in a rear portion, a rear end of the pressing member being connected to the actuator using a wire, and the pressing member integrally having a cutting blade in a front portion and a pressing portion in a rear portion, wherein the third module holds or cuts the suturing thread from front and back in response to the actuator.

8. An endoscopic suturing device for suturing a surgical site in a human body using an endoscope,
the device comprising a position control means inside a tubular body which is insertable into the human body, the position control means controlling upward and downward rotation and forward, backward, leftward and rightward tilting toward a suturing site, wherein the position control means includes a first lever, a second lever and a third lever which are connected together, the first lever being axially coupled to a motor shaft of a first motor which is horizontally disposed on a central cylinder so as to rotate upward and downward, the second and third levers being axially coupled to motor shafts of second and third motors which are vertically disposed and being positioned so as to perpendicularly cross each other, the second and third levers tilting forward and backward and to left and right,
wherein the first module is configured such that a rear end of the needle and one section of a lever are connected in a lower section of the cylinder so as to be rotatable about a hinge, the needle having a pinhole in a leading end through which a suturing thread is inserted, and that the other section of the lever and an actuator in an upper portion of the cylinder are connected to each other using a wire, so that the needle sutures the suturing site while being rotated about the hinge by the actuator.

9. An endoscopic suturing device for suturing a surgical site in a human body using an endoscope,
the device comprising a position control means inside a tubular body which is insertable into the human body, the position control means controlling upward and downward rotation and forward, backward, leftward and rightward tilting toward a suturing site, wherein the position control means includes a first lever, a second lever and a third lever which are connected together, the first lever being axially coupled to a motor shaft of a first motor which is horizontally disposed on a central cylinder so as to rotate upward and downward, the second and third levers being axially coupled to motor shafts of second and third motors which are vertically disposed and being positioned so as to perpendicularly cross each other, the second and third levers tilting forward and backward and to left and right,
wherein the second module comprises a fixing member and a pressing member which are disposed with an “X” shape in a lower portion of the cylinder, a rear end of the pressing member being connected to the actuator using a wire, and middle portions of the fixing member and the pressing member being hinge-coupled to each other, wherein the second module holds a suturing thread in response to the actuator.

10. An endoscopic suturing device for suturing a surgical site in a human body using an endoscope,
the device comprising a position control means inside a tubular body which is insertable into the human body, the position control means controlling upward and downward rotation and forward, backward, leftward and rightward tilting toward a suturing site, wherein the position control means includes a first lever, a second lever and a third lever which are connected together, the first lever being axially coupled to a motor shaft of a first motor which is horizontally disposed on a central cylinder so as to rotate upward and downward, the second and third levers being axially coupled to motor shafts of second and third motors which are vertically disposed and being positioned so as to perpendicularly cross each other, the second and third levers tilting forward and backward and to left and right,
wherein the third module comprises a fixing member and a pressing member which are disposed with an “X” shape in a lower portion of the cylinder, the fixing member integrally having a fixing blade in a front portion and a fixing portion in a rear portion, a rear end of the pressing member being connected to the actuator using a wire, and the pressing member integrally having a cutting blade in a front portion and a pressing portion in a rear portion, wherein the third module holds or cuts a suturing thread from front and back in response to the actuator.

11. The endoscopic suturing device of claim 9, wherein each of a pressing member of a second module and a pressing portion of a pressing member of a third module has a pressing piece in order to elastically hold the suturing thread, the pressing piece being elastically forced by a spring so as to protrude from a recess.

12. The endoscopic suturing device of claim 10, wherein each of a pressing member of a second module and a pressing portion of a pressing member of a third module has a pressing piece in order to elastically hold the suturing thread, the pressing piece being elastically forced by a spring so as to protrude from a recess.
